(19) United States
(12) Patent Application Publication
(10) Pub. No.: US 2007/0203141 A1
(43) Pub. Date: Aug. 30, 2007

(54) METHOD FOR ADMINISTERING BIRB 796 BS

(75) Inventors: Peter M. Grob, New Fairfield, CT (US); Jeffrey B. Madwed, Trumbell, CT (US); Christopher Pargellis, West Redding, CT (US); Chan Loi Yong, Brookfield, CT (US)

Correspondence Address: MICHAEL P. MORRIS;BOEHRINGER INGELHEIM CORPORATION, 900 RIDGEBURY RD, P. O. BOX 368, RIDGEFIELD, CT, 06877-0368, US

(73) Assignee: BOEHRINGER INGELHEIM PHARMACEUTICALS, INC., Ridgefield, CT (US)

(21) Appl. No.: 11/742,975

(22) Filed: May 1, 2007

Related U.S. Application Data
(63) Continuation of application No. 10/313,667, filed on Dec. 6, 2002, now abandoned.
(60) Provisional application No. 60/339,249, filed on Dec. 11, 2001.

(51) Int. Cl. A61K 31/5375

(52) U.S. Cl. 514/237.8

(57) ABSTRACT

Disclosed are methods of administering BIRB 796 BS, a p38 MAPK inhibitor, at particular dosages.

### APPLICATION DATA

This application claims benefit to U.S. provisional application Ser. No. 60/339,249 filed Dec. 11, 2001 and is a continuation of U.S. patent application Ser. No. 10/313,667 filed Dec. 6, 2002, the entirety of which is incorporated by reference herein.

### FIELD OF THE INVENTION

The invention relates to the field treating cytokine mediated diseases.

### BACKGROUND OF THE INVENTION

p38 MAPK is an integral enzyme necessary for the generation of many pro-inflammatory cytokines, eg., TNFα in vitro and in vivo. Inhibitors of this enzyme would therefore be useful in treating cytokine mediated diseases. A potent inhibitor of this enzyme, BIRB 796 BS, is described in U.S. Pat. No. 6,319,921, example no. 8. In the section of the patent describing methods of therapeutic use, it is disclosed that dosage levels may range from about 10-1000 mg/dose for a 70 kg patient, from one dose per day to up to 5 doses per day, for oral doses, up to 2000 mg/day. U.S. application Ser. No. 09/902,822 describes oral formulations of BIRB 796 BS, and U.S. application Ser. No. 10/214,782 provides for parental formulations of the compound.

In-house testing has suggested that BIRB 796 BS is a p38 MAPK inhibitor both in vitro and in vivo.

### SUMMARY OF THE INVENTION

It is an object of the invention to provide a method of administering BIRB 796 BS to a patient in need thereof comprising administering BIRB 796 BS twice daily, each dosage being less than 150 mg of the active ingredient compound. Further embodiments are described in the detailed description of the preferred embodiments.

### DRAWINGS

FIG. 1. Plot of pre-LPS challenge BIRB 796 BS plasma concentrations versus TNFα Percent Inhibition with Predicted Curve Resulting from the Eₘₐₓ Model.

### DETAILED DESCRIPTION OF THE PREFERRED EMBODIMENTS

All terms as used herein in this specification, unless otherwise stated, shall be understood in their ordinary meaning as known in the art.

The term “patient” refers to a warm-blooded mammal and preferably, a human, requiring treatment or prevention of a cytokine mediated disease as described in U.S. application Ser. No. 10/269,173 incorporated herein by reference.

Cytokine mediated diseases include inflammation, acute and chronic pain, from acute and chronic inflammation in the lung caused by inhalation of smoke, endometriosis, Behcet's disease, uveitis and ankylosing spondylitis, pancreatitis, Lyme disease, contact dermatitis, atherosclerosis, glomerulonephritis, reperfusion injury, bone resorption diseases, asthma, stroke, myocardial infarction, thermal injury, adult respiratory distress syndrome (ARDS), multiple organ injury secondary to trauma, dermatoses with acute inflammatory components, acute purulent meningitis, necrotizing entrerocolitis, syndromes associated with hemodialysis, septic shock, leukopherisis granulocyte transfusion, restenosis following percutaneous transluminal coronary angioplasty, Alzheimer's disease, traumatic arthritis, sepsis, chronic obstructive pulmonary disease (COPD), congestive heart failure, rheumatoid arthritis (RA), multiple sclerosis, Guillain-Barre syndrome, Crohn's disease, ulcerative colitis, psoriasis, graft versus host disease, systemic lupus erythematosus, insulin-dependent diabetes mellitus, toxic shock syndrome, osteoarthritis, diabetes and inflammatory bowel diseases. Preferred cytokine mediated diseases include rheumatoid arthritis, Crohn's disease and psoriasis.

WO 01/01986 discloses particular compounds alleged to having the ability to inhibit TNFα. Certain compounds disclosed in WO 01/01986 are indicated to be effective in treating the following diseases: dementia associated with HIV infection, glaucoma, optic-neuropathy, optic neuritis, retinal ischemia, laser induced optic damage, surgery or trauma-induced proliferative vitreoretinopathy, cerebral ischemia, hypoxia-ischemia, hypoglycemia, domoic acid poisoning, anoxia, carbon monoxide or manganese or cyanide poisoning, Huntington's disease, Alzheimer's disease, Parkinson's disease, meningitis, multiple sclerosis and other demyelinating diseases, amyotrophic lateral sclerosis, head and spinal cord trauma, seizures, convulsions, olivopontocerebellar atrophy, neuropathic pain syndromes, diabetic neuropathy, HIV-related neuropathy, MERRF and MELAS syndromes, Leber's disease, Wernicke's encephalophathy, Rett syndrome, homocysteinuria, hyperprolinemia, hyperhomocysteinemia, nonketotic hyperglycinemia, hydroxybutyric aminoaciduria, sulfite oxidase deficiency, combined systems disease, lead encephalopathy, Tourett's syndrome, hepatic encephalopathy, drug addiction, drug tolerance, drug dependency, depression, anxiety and schizophrenia. WO 01/19322 discloses use of p38 inhibitors for treating the common cold or respiratory viral infection caused by human rhinovirus, enteroviruses, coronaviruses, influenza virus, parainfluenza virus, respiratory syncytial virus and adenoviruses. Particular diseases related to such viral infections are asthma, chronic bronchitis, COPD, otitis media, sinusitis and pneumonia. Treating these diseases and conditions are also within the scope of the invention.

In one embodiment, there is provided a method of administering BIRB 796 BS to a patient in need thereof comprising administering BIRB 796 BS twice daily, each dosage being less than 150 mg of the active ingredient compound.

In another embodiment, there is provided a method of administering BIRB 796 BS to a patient in need thereof comprising administering BIRB 796 BS twice daily, each dosage being between 4 and 100 mg of the active ingredient compound.

In yet another embodiment, there is provided a method of administering BIRB 796 BS to a patient in need thereof comprising administering BIRB 796 BS twice daily, each dosage being 4, 5, 15, 30, 45, 60, 75 or 100 mg of the active ingredient compound.

In yet another embodiment, there is provided a method of administering BIRB 796 BS to a patient in need thereof comprising administering BIRB 796 BS twice daily, each dosage being 30, 50, 60, 70 or 90 mg of the active ingredient compound.

In yet another embodiment, there is provided a method of administering BIRB 796 BS to a patient in need thereof comprising administering BIRB 796 BS twice daily, each dosage being 50 or 70 mg of the active ingredient compound.

In yet another embodiment, there is provided a method of administering BIRB 796 BS to a patient in need thereof comprising administering BIRB 796 BS twice daily, each dosage being 50, 60, 70 or 90 mg of the active ingredient compound.

In yet another embodiment, there is provided a method of administering BIRB 796 BS to a patient in need thereof comprising administering BIRB 796 BS twice daily, each dosage being 30, 50 or 70 mg of the active ingredient compound.

As the skilled artisan will appreciate, lower or higher doses within the range provided in this application may be required depending on particular factors. For instance, specific dosage and treatment regimens will depend on factors such as the patient's general health profile, the severity and course of the patient's disorder or disposition thereto, and the judgment of the treating physician.

Routes of administration include, but are not limited to, intravenously, intramuscularly, subcutaneously, intrasynovially, by infusion, sublingually, transdermally, orally, topically or by inhalation. The preferred modes of administration are oral and intravenous. Most preferred is oral.

Dosage forms of BIRB 796 BS include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known. Reference in this regard may be made to H. C. Ansel and N. G. Popovish, Pharmaceutical Dosage Forms and Drug Delivery Systems, 5th ed., Lea and Febiger (1990). Preferred formulations are to be found in Cappola et al. U.S. application Ser. No. 09/902,822 and U.S. application Ser. No. 10/214,782.

In order that this invention be more fully understood, the following examples are set forth. These examples are for the purpose of illustrating preferred embodiments of this invention, and are not to be construed as limiting the scope of the invention in any way.

The Dosage of BIRB 796 BS According to the Embodiments Described Herein was Determined as Follows:

As reported in Arthritis and Rheumatism (Vol 44:9, suppl September 2001, Abstract #671, pg S164) and described below, TNFα production was inhibited by 97% and 88% at 600 and 50 mg of BIRB 796 BS compared to placebo. Also demonstrated below, the EC₅₀ for inhibiting TNFα production in vivo is 23.72 ng/ml, which is significantly lower than (1) that calculated from ex-vivo inhibition in the single dose rising trial (1228 ng/ml, U00-1627), and (2) more importantly significantly lower than the Cmax of 109±51 ng/ml at 15 mg and 208±109 ng/ml at 30 mg on drug day 14 PM that were observed in the BID 14 day Phase 1 trial. In addition, from the Phase 1 single dose trial, the Cmax for the 4 mg dose of BIRB 796 BS was 23.8±5.71 ng/ml. Thus, 4 mg would be efficacious in inhibiting TNFα production.

Finally, from the Phase 1 multi-dose trial, subjects receiving 150 mg BIRB 796 BS daily for seven days had moderate elevations in AST and ALT. Transaminase levels peaked 2 to 4 days after the end of drug therapy on day 7 with resolution over the next 7 to 14 days. The 150 mg BIRB 796 BS dose group also had a small increase in γ-GT. Because of these findings, 150 mg BIRB 796 BS for seven days was not considered well tolerated. Doses of 20 and 50 mg daily for 7 days were well tolerated with no adverse events reported and no laboratory safety abnormalities other than a small increase in ALT and AST in 2 of 6 subjects receiving 50 mg BIRB 796 BS.

Therefore, it is predicted that a dose of less than 150 mg, preferably a dose range of 4-150 mg, would inhibit TNFα production greater than 50% and thus would lead to an efficacious therapeutic dose for RA, Crohns, etc.

Suppression of P38 Activity in Vitro and TNFα Production in Vivo with BIRB 796 BS

BIRB 796 BS attenuates the release of TNFα from LPS stimulated human PBMC (IC₅₀ 21 nM), as well as human and monkey whole blood (IC₅₀ 0.8 uM and 4 uM, respectively). To address the pharmacologic properties of our p38 MAPK inhibitor, we evaluated BIRB 796 BS in two in vivo models of TNFα production. In a mouse model of LPS-induced TNFα production, BIRB 796 BS significantly inhibited TNFα with an ED₅₀ of approximately 10 mg/kg when dosed orally 30 minutes prior to LPS challenge. As a surrogate model for man, we developed an endotoxin model in the cynomolgus monkey to examine the effect of p38 MAPK inhibitors on modulating TNFα production. BIRB 796 BS (0.3, 1 or 3 mg/kg, IV) was administered just prior to LPS challenge (400 ng/kg, IV) in anesthetized male monkeys. BIRB 796 BS significantly inhibited plasma TNFα production by 85% (p<0.005, n=4) and 90% (p<0.005, n=4) at 1 and 3 mg/kg, respectively compared to vehicle treated control animals. TNFα production was not significantly inhibited at the 0.3 mg/kg dose. In a separate experiment to investigate duration of action, BIRB 796 BS (1, 3 or 20 mg/kg, PO, n=4/group) or placebo (n=6) was administered as chewable tablets, 12 hours prior to IV LPS challenge. Compared to placebo, BIRB 796 BS inhibited TNFα production by 44% (NS), 61% (p<0.05) and 84% (p<0.01) with peak plasma levels of 0.003, 0.02 and 1.4 uM for the 1, 3 and 20 mg/kg groups, respectively. These data demonstrate that BIRB 796 BS inhibits TNFα production significantly in vitro and in vivo, showing extended efficacy up to 12 hours after oral dosing in cynomolgus monkeys. This efficacy occurred with blood levels of compound considerably less than the whole blood IC₅₀ suggesting possible partitioning of BIRB 796 BS into tissue sites important for TNFα production. In the clinical endotoxin trial described below, comparable TNFα inhibitory results were observed suggesting our cynomolgus monkey model is a strong predictor of the human cytokine response to LPS.

Anti-Inflammatory Effects of a P38 Mitogen Activated Protein Kinase Inhibitor (BIRB 796 BS) During Human Endotoxemia.

Intravenous administration of endotoxin represents a safe, well-defined model of acute inflammation in humans. It is also an excellent tool to study the mechanisms contributing to inflammatory responses in man in vivo. Given the importance of the balance of inflammatory and anti-inflammatory cytokines and other factors in the etiology of inflammatory diseases such as rheumatoid arthritis and Crohn's disease, administration of BIRB 796 BS in a human LPS model could prove beneficial in elucidating potential effects of BIRB 796 BS in human inflammatory processes.

The primary objective was to examine the effects of BIRB 796 BS on TNFα production in human volunteers challenged with endotoxin. The endotoxin (LPS) was obtained from: Escherichia coli LPS (lot G; United States Pharmacopoeial Convention, Rockville, Md. USA).

When administered orally, 3 hours prior to LPS challenge, BIRB 796 BS inhibited LPS-induced TNFα production by 88% and 97% at 50 and 600 mg, respectively. These data are in accordance with animal data, more specifically, a LPS-induced TNFα production model in cynomolgus monkeys (U98-3153, U99-3145, U99-3034). It also appeared that BIRB 796 BS inhibited its target p38 MAPK because the increase in phosphorylation of p38 MAPK observed with placebo controls was attenuated. The relationship between the percent inhibition and the pre-challenge plasma BIRB 796 BS concentration can be described by an Eₘₐₓ model.

Modelling

Inhibition of Peak Pharmacodynamic Endpoint Values and Pre-LPS Challenge BIRB 796 BS Plasma Concentration Relationship: Nonlinear least-squares regression analysis was performed using BIRB 796 BS plasma concentrations ten minutes prior to the in vivo LPS challenge and percent inhibition of peak TNFα plasma concentration values observed from subjects receiving active BIRB 796 BS treatment. Percent inhibition was obtained by calculating the percent difference between the median peak plasma pharmacodynamic endpoint concentrations of the placebo group and the maximum endpoint concentration achieved by each individual. In this manner, the relationship between the BIRB 796 BS concentrations at the time of the in vivo challenge and the plasma peak level inhibition of the TNFα was examined. The results of the curve fitting showed that the Eₘₐₓ model was adequate in describing the relationship shown in Equation 1.

$$E = \frac{{E}_{max} \cdot C}{{EC}_{50} + C} \qquad Equation 1$$

The following table provides a summary of model parameter values obtained for TNFα:

Pharmacodynamic Parameters Obtained from the Eₘₐₓ Modeling

| | Eₘₐₓ | Eₘₐₓ | EC₅₀ | EC₅₀ |
|---|---|---|---|---|
| | (% Inhibition) | % CV | (ng/mL) | % CV |
| TNFα | 95.34 | 3.28 | 23.72 | 28.13 |

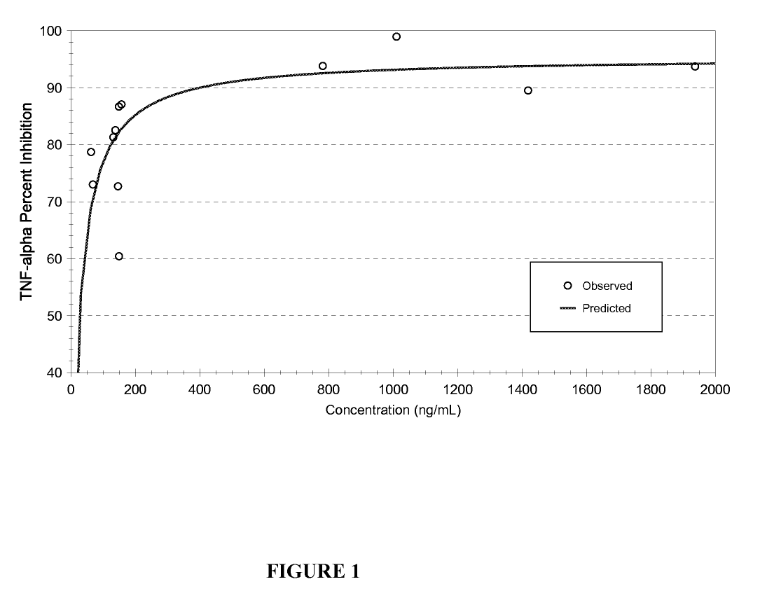

For the primary endpoint, TNFα, BIRB 796 BS exhibited an Eₘₐₓ value of 95% with a low pre-challenge EC50 of 23.72 ng/ml. A graph showing the observed values and the predicted curve from the model is shown in FIG. 1.

Surprisingly, in this endotoxin trial, BIRB 796 BS at 50 mg inhibited TNFα production in vivo even though no inhibition was observed ex vivo in the single dose rising trial (U00-1627). In addition, plasma levels of BIRB 796 BS at 50 mg in this endotoxin trial were considerably lower than the IC₅₀ described for TNFα inhibition in-vitro (U99-3116). Finally, the EC50 calculated from the Emax model in this endotoxin trial was 23.72 ng/ml, which is significantly lower than that calculated from ex-vivo inhibition in the single dose rising trial (1228 ng/ml, U00-1627). This discrepancy between in vivo and ex vivo and in vivo and in vitro was also observed in cynomolgus monkeys (U99-3145). Taken together, these data suggest that the volume of distribution of BIRB 796 BS is appreciable and that compartmentalisation of BIRB 796 BS into tissue, where the main source of TNFα is generated, appears likely.

Safety, Pharmacokinetics and Pharmacodynamics of an Oral Dosage of BIRB 796 BS, Administered Once Daily for 7 Days.

This was a randomized, double-blind, placebo-controlled (2:6 ratio per dose group), multiple dose (20, 50 and 150 mg) study to assess pharmacokinetics, pharmacodynamics, safety and tolerability using a tablet formulation administered once a day for one week. Safety was determined by adverse events, laboratory tests, and physical examination. Kinetics were assessed by measuring plasma levels of drug and pharmacodynamics was assessed ex vivo by the effects of LPS induced production of TNFα, and the TNFα or fMLP induced activation state of PMN in human whole blood as measured by the ratio of Mac-1/Lselectin cell surface expression. Twenty-four healthy male volunteers (mean age 30 years, mean weight 76 kg) were studied. The drug was well tolerated up to 50 mg dose. Two subjects receiving the 150 mg dose developed acne. There were no clinically significant changes in laboratory assessments except for a dose related, reversible rise in AST and ALT. The increase in transaminases was observed in all 6 subjects with the 150 mg dose (up to a 2-fold rise in AST and up to a 3.5-fold rise in ALT above the upper limit of normal). This transaminase increase was not associated with changes in other liver function tests, nor was it associated with any liver related signs or symptoms. Two subjects receiving the 50 mg dose had a much lower, asymptomatic, transient increase in AST or ALT. One subject on the 20 mg dose had a minimal increase in ALT only just above the upper limit of normal. All other safety parameters including ECGs, vital signs and physical examination showed no change with treatment. The pharmacokinetic assessment showed good systemic exposure to the drug with a mean Tₘₐₓ of 1 to 2.25 hours and a plasma t_{1/2} of 7.6 to 9.1 hours. Steady-state was attained within 2 days. Day 7 mean Cₘₐₓ and AUC₀₋₂₄ observed for the three doses were as follows: 20 mg (116 ng/ml, 364ng·hr/ml), 50 mg (308 ng/ml, 1324 ng·hr/ml), and 150 mg (1108 ng/ml, 5924 ng·hr/ml). No inhibition of TNFα was observed at any of the doses used. The drug exhibited a dose dependent inhibition of neutrophil activation 4 hours post dosing, but at 24 hours post dosing, the drug demonstrated a mixed pattern of neutrophil inhibition and activation at the various dose levels. In conclusion, this p38 MAPK inhibitor is orally bioavailable, well tolerated following multiple dose administration up to 50 mg and inhibits ex vivo neutrophil activation 4 hours after administration at doses of 50 mg or higher.

Safety and Pharmacokinetics of an Oral Dosage of BIRB 796 BS, Administered Twice Daily for 14 Days to Healthy Volunteers

This study was a Phase I, randomized, double-blind, placebo-controlled, multiple-dose trial to investigate the safety and pharmacokinetics of 15 or 30 mg of an orally available p38MAPK inhibitor administered twice daily compared to placebo for 14 days. Subjects were 49 healthy males, 16 per treatment group (one subject on placebo was discontinued). A previous study with this drug at doses of 20, 50 and 150 mg once daily for one week showed a reversible, asymptomatic, dose-related rise in ALT and AST primarily with the 150 mg dose. Doses up to 50 mg QD for one week were well tolerated. Based on preliminary analysis of this study, 9 of 48 subjects had transaminase values above the upper limit of normal (UNL), 2 in the placebo, 3 in the 15 mg dose group and 4 in 30 mg dose group. Three subjects experienced ALT rises 2-3 fold greater than UNL, one in each dose group. One subject in the 15-mg dose group had a transient elevation of AST of one-day duration. None of the other subjects who had ALT rises had concurrent elevations of AST or bilirubin (except one subject on the 30-mg dose that had a transient increase in total bilirubin at same time as ALT), and all subjects remained asymptomatic. There were no relevant changes in other laboratory studies, EKGs or physical examinations. Eight subjects (three each in placebo and 30 mg bid groups and two in 15 mg bid group) had a total of 16 adverse events in 10 overall episodes, none of which were serious or considered drug related. The pharmacokinetic assessment showed good systemic exposure to drug with a Cₘₐₓ and AUC0-12 (mean±SD) on day 14 of 109±51 ng/ml and 334±145 ng·hr/ml (15 mg); and 208±109 ng/ml and 659±449 ng·hr/ml (30 mg), respectively. Dose proportionality was observed for both Cmax and AUC0-12. Mean elimination half-life was 7.3 hr. Based on these results, this oral p38MAPK inhibitor exhibits good pharmacokinetic profiles when administered twice daily at 15 and 30 mg, and is well tolerated at doses up to 30 mg twice daily for two weeks.

All references cited in this application are incorporated herein by reference in their entirety.

We claim:

1. A method of administering BIRB 796 BS to a patient in need of treatment of psoriasis comprising administering BIRB 796 BS twice daily, each dosage being less than 150 mg of the active ingredient compound.

2. The method according to claim 1 wherein each dosage is between 4 and 100 mg of the active ingredient compound.

3. The method according to claim 1 wherein each dosage is 4, 5, 15, 30, 45, 60, 75 or 100 mg of the active ingredient compound.

4. The method according to claim 2 wherein each dosage is 30, 50, 60, 70 or 90 mg of the active ingredient compound.

5. The method according to claim 4 wherein each dosage is 30, 50 or 70 mg of the active ingredient compound.
